(19) United States
(12) Patent Application Publication
(10) Pub. No.: US 2005/0222494 A1
(43) Pub. Date: Oct. 6, 2005

(54) Dual-scope colonoscopy system with separate secondary colonoscope tool

(76) Inventor: James T. Prescott, McPherson, KS (US)

Correspondence Address: SHERMAN D PERNIA, ESQ., PC, 1110 NASA ROAD ONE, SUITE 450, HOUSTON, TX, 77058-3310, US

(21) Appl. No.: 11/075,011

(22) Filed: Mar. 8, 2005

Related U.S. Application Data
(63) Continuation-in-part of application No. 10/818,768, filed on Apr. 6, 2004.

(51) Int. Cl. A61B 1/018

(52) U.S. Cl. 600/113; 600/153; 600/106

(57) ABSTRACT

The present duel-scope colonoscopy system has a primary or parent colonoscope substantially typical of the field, but adapted to incorporate a separate, extended reach secondary or auxiliary colonoscope tool. The insertion tube of the secondary colonoscope tool is received in a tool channel of the parent scope, and is “extended reach” in that the secondary insertion tube is extendable from the tip of the primary insertion tube of the parent scope an additional distance beyond the reach of the primary scope tube. The secondary colonoscope tool is a separate and complete image viewing fiberoptic colonoscope having a flexible, small diameter fiberoptic cable insertion tube of a length sufficient to substantially extend beyond a distal end the insertion tube of the parent endoscope.

### FIELD OF THE INVENTION

The present invention is in the field of colonoscopes having a generally tube-like structure with a proximal operating end and a distal inserting end, means to transmit a view or image from the distal end to the proximal end, and a light source to transmit light from the proximal end to the distal end, wherein the distal end is inserted into a natural body orifice for viewing or treating the body. Specifically, the present invention relates to a parent colonoscope provided with an additional, smaller viewing means for enabling two separate views through the colonoscope. Additionally, the present invention relates to an insertion means to move the smaller viewing means through an auxiliary channel on the parent colonoscope, and to the smaller viewing means having a particular distal tip lens configuration.

### BACKGROUND OF THE INVENTION

Colonoscopy is a medical procedure in which a physician looks inside a patient's entire large intestine, from the lowest part (the rectum) all the way up through the colon to the lower end of the small intestine (the cecum). The procedure is used to search for early signs of cancer in the colon and rectum. It is also used to diagnose the causes of unexplained changes in bowel habits. Colonoscopy enables the physician to see inflamed tissue, abnormal growths, ulcers, and bleeding. In colonoscopy, depending upon the skill of the physician/operator, roughly 5 to 20 percent of the cases are not fully completed due to an inability to advance the tip of the colonoscope's insertion tube to the cecum of the lower intestines. If a colonoscopy procedure is unable to be fully completed, the alternative is expensive additional testing, such as barium enema virtual colonoscopy with a CT scanner.

Additionally, in the cases that are fully completed, a substantial amount of time is often spent manipulating the insertion tube trying to advance the tip of the insertion tube the last foot or so of the procedure. This manipulation not only takes additional time, but exposes the patient to the increased risk associated with prolonging anesthesia, as well as increasing the risk of perforation (not to mention the discomfort) due to repeated withdrawal and advance of the insertion tube. This problem has existed and been recognized in the field as long as current colonoscopes have been in service, but still no completely satisfactory solution is available.

A solution to the problem of uncompleted colonoscopy due to limitation of reach of current colonoscope insertion tubes could be solved in certain cases if there was a way to advance just the imaging means of the scope beyond the tip of the insertion tube. This is not possible with the imaging means of current colonoscopes, which are substantially fixed in relationship to the insertion tube. However, the combination of the current primary colonoscope with a secondary imaging means separate from and not fixed in relationship to the primary colonoscope's insertion tube, which imaging means can be advanced beyond the tip of the primary insertion tube would be a solution. In other words, the combination of two separate colonoscopes.

Generally, endoscopes of various types have been used in different medical fields for many years. The types of fiberoptic medical endoscopes currently available vary widely depending on their application, from having relatively short, small diameter insertion shafts (e.g., certain laparoscopes and bronchoscopes) to having relatively large shaft diameters and lengths (e.g., colonoscopes). Likewise, the field has been motivated to provide endoscopes having more than one means of viewing the same image, as well as endoscopes having means for viewing more than one image. However, to view more than one image, the scope requires an additional viewing means to be able to view a different image.

For example, Carpenter (U.S. Pat. No. 4,586,491) discloses a bronchoscope with the usual and customary working/tool channels, where one of the channels is adapted to receive a small gauge catheterscope. The catheterscope enables a user of the Carpenter bronchoscope to view two different images. The catheterscope is passed through the adapted working channel to reach beyond the parent scope and view more remote small diameter bronchioli. The diameter of the optic cable of Carpenter's secondary catheterscope is very small: on the order of 0.066 to 0.110 inches. The catheterscope fiberoptic cable in Carpenter is inserted into the parent scope “in conventional fashion,” i.e., by free hand. Extension of the catheterscope optic cable from the parent scope is then limited to the relatively short throw of a piston integral with the catheterscope assembly. However, the distal end of the Carpenter catheterscope cannot be extended any further from the parent scope's distal end than the throw of the piston. Additionally, small diameter fiberoptic cables can be delicate and readily subject to kinking when relatively long sections are being manipulated by hand.

Carpenter also discloses (in U.S. Pat. No. 4,947,828 to Carpenter et al.) piggybacked endoscopes which releseably connect together and receive illumination from a single light source. Although in this device each endoscope has a separate control handle, the optical paths are not movable relative to each other.

Komi (U.S. Pat. No. 4,979,496) discloses a double endoscope system for inspecting the bile and pancreatic ducts comprising a “mother” scope and a “daughter scope. In Komi, a guide tube is inserted into a tool channel on the side of the mother scope. The daughter scope then inserts into the guide tube through an insertion port at the end of the guide tube. The insertion unit of the daughter unit is shorter than the length of the guide tube into which it is inserted (see Komi FIG. 17), and is not extendable from the distal end of the daughter scope. This means that the Komi scope can only be forward looking. The guide tube of Komi is a liner for the tool channel and additionally serves as a conduit for the injection of materials into the site of the distal end of the daughter scope insertion unit.

The daughter scope insertion unit of Komi, containing the fiberoptics is only 0.8 mm in diameter. The combination light path and image path of the Komi device is very small, and is not useable in an application requiring the illumination of a volume of space relatively larger than the bile duct. Additionally, such a small diameter fiberoptic image path is not capable of sufficient image (pixel) resolution in volumes and at focal distances substantially larger than encountered in the bile duct.

Although the devices of the above noted disclosures each may be useful for its intended purpose, it would be useful in the field to have a simpler alternative duel-scope system useful for colonoscopy, having an imaging means capable of reaching beyond the end of the parent scope, of delivering sufficient light and having sufficient image resolution for the spaces under investigation in a colonoscopy. Additionally, it would be useful to have an insertion tube feed mechanism that can provide controlled mechanical movement of small diameter insertion tubes, and have the insertion tubes be less susceptible to kinking from free-hand manipulation during use.

### SUMMARY OF THE INVENTION

It is an object of the present invention to provide a duel-scope colonoscopy system for performing medical colonoscopic procedures. The present duel-scope colonoscopy system has a primary or parent colonoscope substantially typical of the field, but adapted to incorporate a separate, extended reach secondary or auxiliary colonoscope tool. The insertion tube of the secondary colonoscope tool is received in a tool channel of the parent scope, and is “extended reach” in that the secondary insertion tube is extendable from the tip of the primary insertion tube of the parent scope an additional distance beyond the reach of the primary scope tube. The secondary or auxiliary colonoscope tool for use with a parent colonoscope is an apparatus comprising a separate and complete image viewing fiberoptic colonoscope. The secondary colonoscope has a scope body interfacing with a flexible, small diameter fiberoptic cable insertion tube of a length sufficient to substantially extend beyond a distal end the insertion tube of the parent endoscope. Additionally, the secondary colonoscope includes an image viewer, and a light source connector. The image viewer may be either optical or digital.

The auxiliary scope's secondary insertion tube consists primarily of a relatively small diameter fiberoptic cable, including both a light path and an imaging path. The optical fibers of the cable can be very brittle, and even when contained in a cable sheath they can be subject to kinking. This is especially true for thin fiberoptics of relatively long length when being manipulated by hand. Therefore, the present invention includes an optional optical cable insertion mechanism for those applications and embodiments where kinking of the cable is a risk. When used, the cable insertion mechanism received the secondary insertion tube in one end and interfaces with the tool channel on the primary colonoscope at the other end. A spring clamp and slide lever assembly on the mechanism is used to engage the insertion tube and to control its insertion and positioning in the tool channel of the parent colonoscope.

As a further option, a ported coupling can be installed in the path of the entry to the tool channel to provide a means of injecting water (or other solution) or air into the lumen of the colon as is typically done during a procedure. Appropriate couplings are known in the field and are adaptable for use in the present invention by one of ordinary skill in the art. For example, a standard two-way selectable Luer-type T-coupling may be adapted by having a septum port at the fitting on the coupling that receives the insertion tube of the secondary colonoscope.

The secondary colonoscope of the present duel-scope colonoscopy system comprises a scope body which interfaces with the secondary insertion tube, a light source and an image viewer. The secondary insertion tube contains the fiberoptics, is flexible and has sufficient length to allow it to be extended substantially beyond the distal end of primary insertion tube of the parent colonoscope. This extension is intended to be at least six inches, but typically will be about twelve or more inches. At the proximal end of the secondary insertion tube the separate light and image paths of the fiberoptic cable are in communication with the light source and the image viewer respectively. At its distal end, the insertion tube terminates in an insertion tube tip section, where the light path emits light and the imaging path interfaces with a lens assembly.

The image viewer of the secondary colonoscope is disposed to receive and present an optical image communicated by the fiberoptic cable for viewing. The image communicated by the fiberoptic cable is optical in that it is a light image developed by a lens system, and not an electrical signal generated by a digital camera. However, the viewer itself may be either optical or digital.

The lens assembly in the tip section of the secondary insertion tube is disposed to provide a field of view substantially perpendicular to the length of the insertion tube (i.e., the axis of the tip section). This is unusual in colonoscope, and can allow viewing of an image of the intestinal wall less distorted than might be available from the wide angle lens of the primary colonoscope. This alternative view feature is an important adjunct to a colonoscopy system practicable by the present invention. If preferred, the lens assembly of the secondary insertion tube can be disposed to provide a field of view angled to look backward from perpendicular along the length of the secondary insertion tube at the tube tip. This alternative view capability allows an operator to look at the other side of a feature in the intestine that cannot be view on advancement of the parent colonoscope's insertion tube. This capability currently does not exist in the field.

An additional feature of the secondary colonoscope of the present invention is that the imaging path of the fiberoptic cable interfaces directly with a tapered light collector which in turn couples the imaging path to the imaging lens in the tip section of the secondary insertion tube. Because of the small diameter of the secondary insertion tube relative to the parent scope's insertion tube, the secondary fiberoptics are of necessity relatively small and must readily pass through the tool channel of the parent scope. Therefore, it is important to adapt the fiberoptics of the secondary colonoscope to optimize utilization of available light as much as possible. Use of the current tapered light collector allows use of a relatively larger image light collecting lens than the cross-section of the fiberoptic light path might otherwise permit.

Where sufficient lumination and/or resolution in not an issue, the secondary colonoscope can have a secondary tool channel passing from a proximal scope body end of the secondary colonoscope's insertion tube and out the distal end of the secondary insertion tube. This secondary channel can be used in a manner typical for colonoscopes to deliver water or air to a site in the lumen of the large intestine.

### BRIEF DESCRIPTION OF THE DRAWINGS

FIG. 1A is a side elevation view of a secondary endoscope intended for use as the auxiliary endoscope assembly of the present invention, the secondary endoscope having an optical image viewer.

FIG. 1B is a side elevation, partial cross-sectional view of a secondary endoscope intended for use as the auxiliary endoscope assembly of the present invention, the secondary endoscope having a digital camera image viewer.

FIG. 2A is a schematic representation of an exemplary prior art controller housing for the parent endoscope of the present invention, but showing the present mechanical optical cable insertion assembly connectable to a tool channel port on the side of the housing.

FIG. 2B is a schematic representation of an exemplary prior art controller housing for the parent endoscope of the present invention, but showing the present mechanical optical cable insertion assembly installed on a tool channel port on the side of the housing and the optical cable of the secondary colonoscope received in the insertion assembly. Also shown is a T-coupling that may be inserted between the insertion assembly and the tool port to provide a lavage port for pushing a fluid through the tool channel while the secondary optical cable is in place.

FIGS. 3A, 3B and 3C respectively are a first end view, a side view and a second end view of the insertion assembly.

FIGS. 4A, 4B and 4C respectively are a first end view, a side cross-sectional view and a second end view of the insertion assembly.

FIG. 5 is a frontal view of the tip section of the secondary insertion tube showing the light conduits, the image lens and the tool channel.

FIG. 6 is a partial cross-sectional view of the distal end tip of the secondary insertion tube with a side looking lens assembly with a tapered light collecting coupling interfacing between the distal lens and the imaging path of the fiberoptic cable.

### DETAILED DESCRIPTION OF THE INVENTION

Referring now to the drawings, the details of preferred embodiments of the present invention are graphically and schematically illustrated. Like elements in the drawings are represented by like numbers, and any similar elements are represented by like numbers with a different lower case letter suffix.

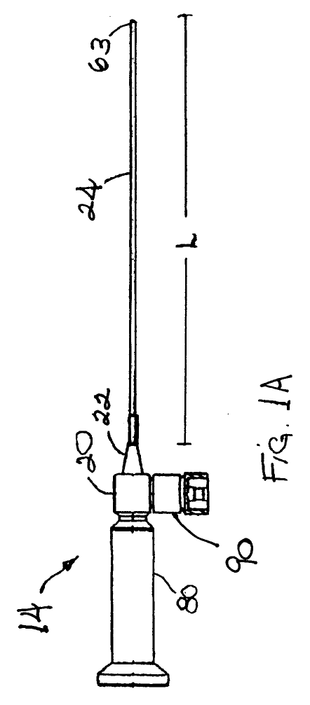

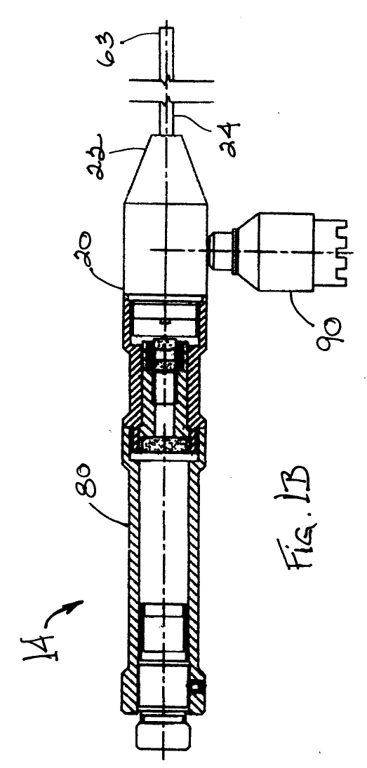

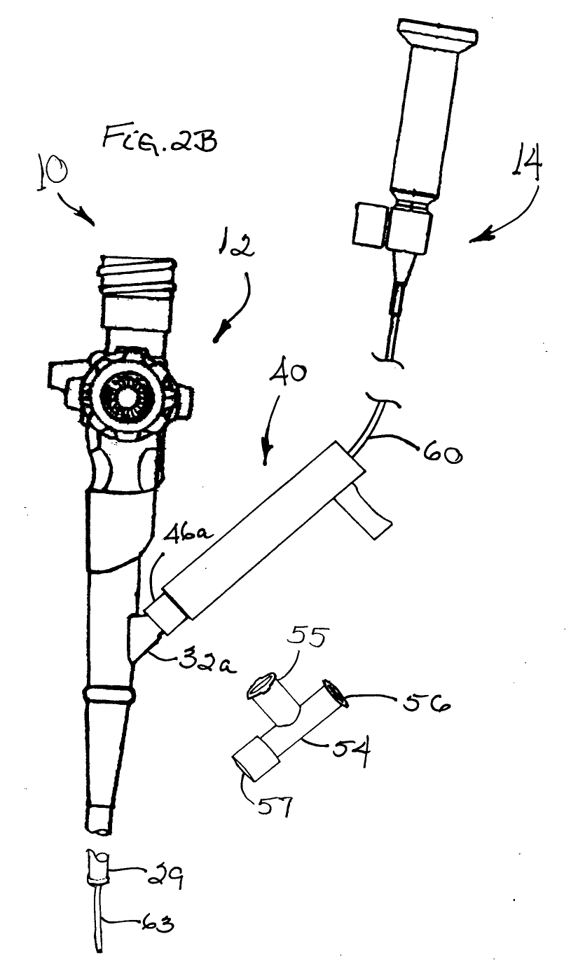

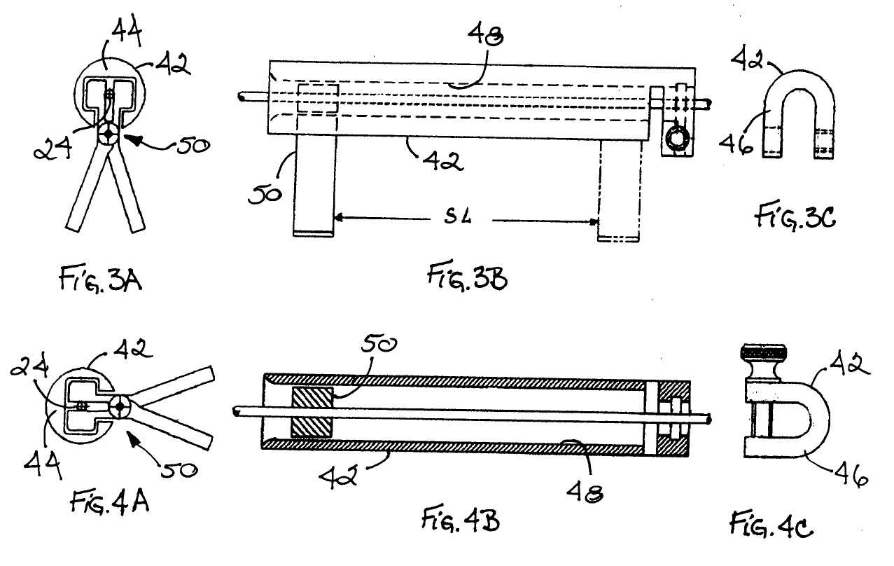

The present duel-scope colonoscopy system 10 is the combination of a primary or parent colonoscope 12 substantially typical of the field, but adapted to incorporate a separate, extended reach secondary or auxiliary colonoscope 14 (see FIG. 3B). The auxiliary or secondary colonoscope 14 for use with the parent colonoscope 12 of the present invention 10 is exemplified in FIGS. 1A and 1B. The main components of the secondary colonoscope 14 include a fiberoptic colonoscope body 20, a flexible fiberoptic cable insertion tube 24 and cable head 22, an image viewer 80 and a light source connection 90. At the distal end of the secondary insertion tube 24 is a tip section 63 housing a specially adapted colonoscope lens assembly.

The extended reach insertion tube 60 of the secondary colonoscope tool 14 is received in a tool channel 30 of the parent scope 12, as shown in FIG. 3B. The secondary insertion tube 60 is “extended reach” in that the distal end of the secondary insertion tube 24 is extendable from the tip section 29 of the primary insertion tube 28 of the parent scope 12 an additional distance beyond the reach of the primary scope tube. The secondary or auxiliary colonoscope tool 14 for use with the parent colonoscope 12 is an apparatus comprising a separate and complete image viewing fiberoptic colonoscope. The secondary colonoscope 14 has a secondary fiberoptic cable insertion tube 24 of a length L sufficient to extend substantially beyond the primary tip section 29 at the distal end of the insertion tube 28 of the parent colonoscope 12. The image viewer 80 of the secondary colonoscope 14 can be either optical or digital.

In a preferred embodiment, the present duel-scope colonoscopy system 10 includes an insertion tube feed mechanism 40 (see FIGS. 3A and 3B). The insertion tube feed mechanism 40 is connectable to the tool channel port 32. The feed mechanism 40 allows the controlled feeding of the secondary insertion tube 24 into the tool port 32 of the primary tool channel 30. Also, the feed mechanism 40 allows controlled extension of the tip section 63 of the secondary tube 24 from the distal end 29 of the parent scope insertion tube 28.

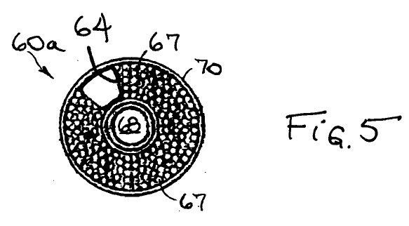

In the preferred embodiment illustrated in FIGS. 3A to 3C and FIGS. 4A to 4C, the insertion tube feed mechanism 40 comprises a slotted housing 42 having a first end 44 and a second end 46 and a through passage 48 connecting the ends. The first end 44 of the slotted housing 42 and passage 48 being adapted to initially receive the distal end tip section 63 of the secondary insertion tube 24, and the second end 46 of the slotted housing 42 and passage 48 being adapted to pass the secondary insertion tube 24 from the insertion mechanism 40. A cable drive mechanism 50 is disposed within the housing 42 for drawing the fiberoptic cable insertion tube 24 through the feed mechanism 40. In the embodiment illustrated, the cable drive mechanism comprised a pinch clamp/slide assembly 50 slidably retained in the through passage 48 of the housing 42.

In use, the pinch clamp/slide assembly 50 of the feed mechanism 40 is used to engage and grip the secondary insertion tube 24 proximate the first end 44 of the housing 42. Then the pinch clamp/slide assembly 50 is slid along the through passage 48 of the slotted housing 42 toward the second end 46 of the feed mechanism 40 to mechanically feed the secondary insertion tube 24 into the tool channel 30. Exact positioning of the secondary insertion tip section 63 of the secondary insertion tube 24 relative to the parent insertion tube tip section 29 of the parent colonoscope 12 is controllable by appropriately sliding the pinch clamp/slide assembly 50 in the desired distance along its stroke length SL.

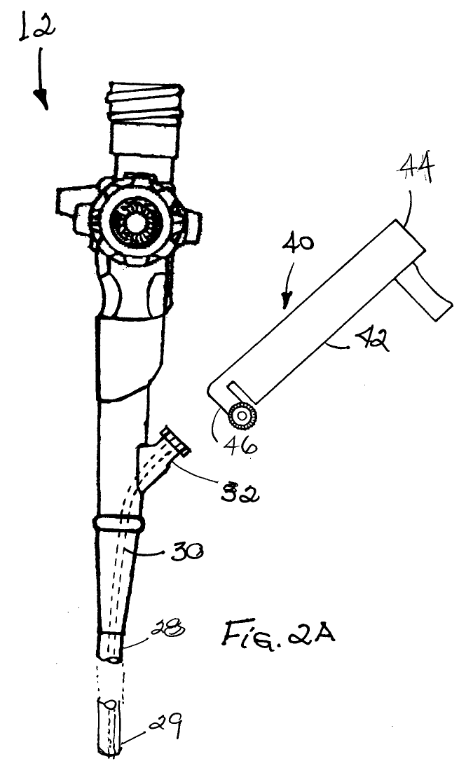

The second end 46 of the feed mechanism 40 is a tool channel interface. The tool channel interface 46 is configured to attach feed mechanism 40 to the tool channel port fitting 32 on the parent colonoscope 12. Additionally, the tool channel interface 46 aligns the through passage 48 of the feed mechanism 40 with the bore of the tool channel 30 on the parent colonoscope. In FIG. 2A and FIGS. 3A to 4C, the tool channel interface 46 comprises a screw clamp which attaches the feed mechanism 40 to the port fitting 32. In FIG. 2B, a leur-lock port fitting 32a and the tool channel interface 46a are complimentary parts of a typical leur lock type connection interface.

Optionally, as illustrated in FIG. 2B, the present duel-scope colonoscopy system 10 can include a ported coupling 54 installable between the feed mechanism 40 and the tool channel port 32a, in the path of the entry to the tool channel 30. The ported coupling 54 can provide a means of injecting water (or other solution) or air through the tool channel 30 and into the lumen of the colon as is typically done during a procedure. The ported coupling 54 has a through bore communicating with a seal or septum port 56 at one end of the through bore and with a port fitting 57 at the other end. An intersecting bore port 55 is also in communication with the through bore of the ported coupling 54. The port each has an appropriate coupling means, like leur fittings. For example, a standard three-way Luer-type T-coupling may be adapted by having a septum port 56 at the inlet fitting on the coupling that receives the insertion tube of the secondary colonoscope. Appropriate couplings are known in the field and are adaptable for use in the present invention by one of ordinary skill in the art.

Optionally, as illustrated in FIG. 5, the secondary insertion tube 60a can be configured to include a secondary tube tool channel 64. The secondary tool channel 64 passes from proximate the scope body 20 of the colonoscope's secondary insertion tube 24 and out the tip of its distal end section 63. In the embodiment illustrated, the secondary tube tool channel 64 shares part of a circumferential area of the insertion tube 60a with the light path conduits 67. In this embodiment, the imaging path 68 is in the center of the cross-section of the insertion tube 60a. Other configurations for including a secondary tube tool channel 64 within the secondary insertion tube 60a are known to and selectable by one of ordinary skill in the art.

It is an important feature of the present duel-scope colonoscopy system 10 that the secondary colonoscope 14 has a secondary insertion tube 24 having a length L sufficient to substantially extend beyond the distal end section 29 of the insertion tube 28 of the parent colonoscope 12. It is preferred that insertion tube 60 of the secondary colonoscope 14 have a length L sufficient to extend at least six inches beyond the distal end 29 of the parent colonoscope 12. Practically speaking, it is preferred that secondary insertion tube 24 be extendable at least about twelve inches beyond the distal end 29 of the parent colonoscope 12.

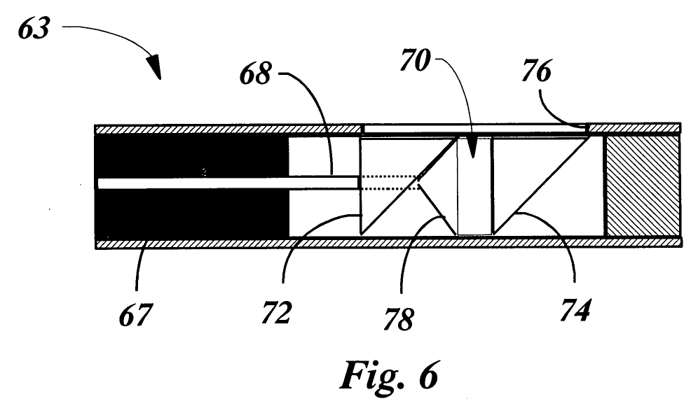

In a preferred embodiment of the secondary scope tool 14 shown in FIG. 6, the fiberoptics terminate in the tip section 63 in an imaging lens assembly 70 having a field of view substantially perpendicular to the length L of the secondary insertion tube 24. Alternatively, the lens assembly 70 can be disposed to provide a field of view angled to look backward from perpendicular along the length of the secondary insertion tube at the tube tip. The benefit these alternative scope views in a secondary colonoscope bring to colonoscopy procedures are noted above.

In the preferred embodiment illustrated in FIG. 6, the side viewing lens assembly 70 of the present invention comprises a pair of prism lenses, one being a light transmitting lens 72 and the other being an imaging lens 74. The prismatic light lense 72 redirects light emitted from the light path conduits 67 out of the side view window 76 in the distal end tip 63. Reflected light passes back through the side view window 76 and enters the prismatic imaging lens 74. The imaging lens in this embodiment interfaces with an optional tapered light collector 78. The tapered light collector 78 couples the imaging lens 74 of the lens assembly 70 to the image path 68 of the fiberoptics at the tip section 63 of the secondary insertion tube 24. The light collector 78 gathers the reflected light from the imaging lense 74 and focuses its communication to the image path 68.

Optionally, a tapered light collector (not shown) can be included in the light coupling 90 to increase the amount of light available from a light source that gets concentrated into the light path 67 of the fiberoptics. This feature may be useful when attaching a low power or portable light source, such as a Welch-Allyn® power handle, to the sight source connection 90.

While the above description contains many specifics, these should not be construed as limitations on the scope of the invention, but rather as exemplifications of one or another preferred embodiment thereof. Many other variations are possible, which would be obvious to one skilled in the art. Accordingly, the scope of the invention should be determined by the scope of the appended claims and their equivalents, and not just by the embodiments.

1. A duel-scope colonoscopy system comprising a parent colonoscope having a primary insertion tube within which a tool channel is disposed; and a secondary colonoscope having a secondary insertion tube which secondary insertion tube is receivable in the tool channel and has a tube length disposed to be extendable beyond a tip of the tool channel.

2. The duel-scope colonoscopy system of claim 1, further comprising an insertion tube feed assembly connectable to the tool channel, the feed assembly for controlling the feeding of the secondary insertion tube into the tool channel and its disposition within the tool channel.

3. The duel-scope colonoscopy system of claim 2, wherein the insertion tube feed assembly comprises:
a slotted tube having a first end and a second end and a through passage connecting the ends;
the first end of the slotted tube and passage being adapted to initially receive a distal end of the insertion tube, and the second end of the slotted tube and passage adapted for passing the insertion tube through the insertion assembly;
a drive mechanism for drawing the fiberoptic cable insertion tube through the insertion tube feed assembly; and
a tool channel interface, the interface disposed at the second end of the feed assembly and configured to mate the through passage of the feed assembly with a bore of the tool channel on the parent colonoscope.

4. The duel-scope colonoscopy system of claim 1, further comprising a ported coupling, the coupling having a through bore and an intersecting bore, with a sealable port at one end of the through bore and open ports at the other ends of the bores of the coupling, with the ports having appropriate coupling means.

5. The duel-scope colonoscopy system of claim 1, wherein the secondary colonoscope comprises:
an optical image viewing fiberoptic colonoscope having a scope body, the scope body interfacing with a secondary insertion tube, a light source and an image viewer;
the secondary insertion tube being flexible and having a length sufficient to substantially extend beyond a distal end of a primary insertion tube of the parent colonoscope, the secondary insertion tube including a flexible fiberoptic cable in light communication at a proximal end with the light source and the image viewer and at a distal end with an insertion tube tip section;
the image viewer disposed to receive and present an optical image communicated by the fiberoptic cable for viewing; and
the fiberoptic tip having a field of view substantially perpendicular to a length of the insertion tube.

6. The secondary colonoscope of claim 5, wherein the light source connector is adapted to attach to a Welch-Allyn® power handle light source.

7. The secondary colonoscope of claim 5, wherein the image viewer is adapted to communicate the optical image to a digital imaging device.

8. The secondary colonoscope of claim 5, wherein the secondary insertion tube has a length sufficient to extend at least six inches beyond the distal end of the primary insertion tube of the parent colonoscope.

9. The secondary colonoscope of claim 5, wherein the flexible fiberoptic cable of the secondary insertion tube includes the insertion tube tip section having an imaging lens with a field of view substantially perpendicular to the length of the secondary insertion tube at the tube tip.

10. The secondary colonoscope of claim 5, wherein the flexible fiberoptic cable of the secondary insertion tube includes the insertion tube tip section having an imaging lens with a field of view angled to look backward from perpendicular along the length of the secondary insertion tube at the tube tip.

11. The secondary colonoscope of claim 5, wherein the flexible fiberoptic cable of the secondary insertion tube includes the insertion tube tip section having tapered light collector coupling the imaging lens to an image path of the fiberoptic cable.

12. The secondary colonoscope of claim 5, wherein the flexible fiberoptic cable of the secondary insertion tube includes the insertion tube tip section having a fiberoptic lens with a field of view perpendicular to the length of the secondary insertion tube at the tube tip.

13. The duel-scope colonoscopy system of claim 5, wherein the secondary colonoscope further comprising the secondary insertion tube having a secondary tool channel passing from a proximal scope body end of the secondary colonoscope's insertion tube and out the distal end of the secondary insertion tube.

14. A duel imaging colonoscopy system comprising:
a parent colonoscope having a tool channel, the tool channel passing from a proximal controller end of the parent colonoscope's insertion tube and out a distal end of the insertion tube, the tool channel being adapted at the controller end to interface with a secondary colonoscope; and
the secondary colonoscope being the secondary colonoscope of claim 1, and having an insertion tube adapted to be received into and to extend beyond the tool channel of the parent colonoscope.
